(19) United States
(12) Patent Application Publication
(10) Pub. No.: US 2018/0126129 A1
(43) Pub. Date: May 10, 2018

(54) Implantable Stent

(71) Applicant: Ardle Tomas McDonough, Galway (IE)

(72) Inventor: Ardle Tomas McDonough, Galway (IE)

(73) Assignee: STENTORIUM LTD, Galway (IE)

(21) Appl. No.: 15/728,500

(22) Filed: Oct. 10, 2017

Related U.S. Application Data
(63) Continuation-in-part of application No. 14/336,822, filed on Jul. 21, 2014, now abandoned.

(51) Int. Cl. A61M 27/00; A61M 25/01; A61M 25/00

(52) U.S. Cl. CPC A61M 27/002; A61M 27/008; A61M 25/0108; A61M 25/0045; A61M 25/09016; A61M 2250/00; A61M 2240/00; A61M 2025/0046; A61M 25/0138

(57) ABSTRACT

The invention is a stent designed for indwelling in a body, where its purpose would be to assist in the drainage of liquid from one part of the body to another. The stent is composed of a material that would typically be coiled to form a cylindrical shape along the length of the stent. Each end of the stent would typically be shaped to form a looped pigtail to prevent migration in the vessel. The stent can be used for minimally invasive procedures or alternatively, it could be placed percutaneously. This stent could be used in various parts of the body, such as the ureter, urethra, bile duct, liver, pancreas, vascular system and neurovascular system.

### RELATED APPLICATIONS

This application is a continuation-in-part of U.S. application Ser. No. 14/336,822 filed Jul. 21, 2014 and titled “Implantable Stent”, the entirety of which is incorporated herein by reference. Any and all applications for which a foreign or domestic priority claim is identified in the Application Data Sheet as filed with the present application are hereby incorporated by reference under 37 CFR 1.57.

### STATEMENT AS TO THE RIGHTS TO INVENTIONS MADE UNDER FEDERALLY SPONSORED RESEARCH AND DEVELOPMENT

This invention was not made with U.S. government support.

### TECHNICAL FIELD

The technical field of this invention is an implantable medical device. This invention is particularly suitable for use in veterinary or paediatric urological procedures.

### BACKGROUND OF THE INVENTION

The placement of urology stents via minimally invasive techniques has continued to increase incrementally over the last decade. Both metal and plastic stents of various designs are being placed.

A ureteral stent, for example, can be placed in certain circumstances to allow urine to drain from the kidney to the bladder and out of the body, when urine cannot flow through the ureter with ease.

Recent advances in veterinary medicine have also enabled the placement of urology stents in animals, however, many, or all, of the urology stents being placed in veterinary medicine at present are plastic stents. These products are generally susceptible to higher levels of encrustation than metal stents because of a reaction between the plastic stent and bacteria in the urinary system. The encrustation can make the stents difficult to remove, which is obviously problematic. For this reason, the recommended indwell duration for human use of these plastic stents is only eight weeks, which is rarely suitable for the intended use in animals.

Another shortcoming with plastic stents of smaller diameters is low radial strength. This is particularly problematic when there is a tight stricture or a stone that cannot be easily removed and where the ureter of the patient is between 0.3 mm and 1.0 mm in diameter. In some cases, the stent is unable to withstand the compressive forces exerted on it and it malfunctions (Docio S G and DeWolf W C, (1989), J Urol, 142 (2 Pt 1):277-9).

A further issue is an inability to produce smaller diameter stents with current stent designs and stent kits. Inserting stents that exceed the size of the vessel can cause unnecessary and irreversible dilation of the vessel. This is an issue where vessels are smaller than any stent available, such as in the ureters or pancreatic ducts of smaller animals, such as cats or small dogs. The result of placing stents that exceed the size of the vessel is that the stent can never be removed, since the vessel would collapse.

Furthermore, since the size of current stents is larger than the vessel for which they are intended, there is difficulty in placing the current stents. Hospital procedures and duration of anaesthesia are therefore longer than necessary.

It would not be possible to use any stent or stent kit previously invented to solve the problems described above. The stent and stent kit of the instant application is a solution to these issues.

Regarding metal ureteral stents, the stents and stent kits identified as prior art in the instant application do not work for smaller locations in the body or for smaller patients such as children.

U.S. Pat. No. 4,813,925 describes a stent with helical coils that do not touch which, in turn, reduces radial strength. A single wire is used which reduces radial strength and in turn reduces holding strength in the looped ends of the stent. This results in a higher chance of deformation and the inability of the looped ends to maintain their place in a stent. This problem is also found in smaller stents where wires are akin to thread used in common sewing. The use of hollow wire to allow for the insertion of a guide wire therein to situate the stent in a desired location is not possible in stents utilizing smaller wire dimensions.

The stent kit described in U.S. Pat. No. 7,550,012 (“the Lavelle patent”) utilizes a hollow wire which is not feasible for smaller stents as wire dimensions decrease due to the loss of wall strength in the wire. Similarly, the delivery system lacks sufficient compressive strength to place a stent in locations of smaller dimensions. The innovative use of a mandrel of high compressive strength in the stent inserter of the present invention solves this problem.

The dome-shaped caps and cannula described in the Lavelle patent would most likely prevent the coil from stretching excessively upon removal of the stent from the patient. The stent of the present invention uses a closely connected coil, which is laser-welded at the ends but which has no cannula connecting the two ends. In terms of design, a cannula is not necessary in this invention to preserve tensile strength since it is generally not possible to remove a ureteral stent minimally invasively by pulling it from the bladder using a cystoscope in small animal and certain instances in paediatric surgery. As such, the usefulness of preventing the coil from stretching is limited in these cases. It is more desirable to push the stent out of the ureter and retrieve it from the bladder, which eliminates the stretching issue. As outlined elsewhere, any longitudinal connection in prior art, as in any portion of solid plastic or metal tube or by adding a cannula or other object, increases the ability to push and pull the stent, but it decreases flexibility. This invention is designed for a specific function in which flexibility for tortuous conditions is key to performance and in which fully minimally invasive surgery is not possible in a variety of cases, reducing the need to pull the stent.

### BRIEF SUMMARY OF THE INVENTION

The unique design of this stent and stent kit allows for the creation of smaller, better functioning stents than are currently capable of being produced quickly and cheaper than the stents currently on the market or described in the prior art.

In the preferred embodiment, the invention is directed to a thick-walled stent, as defined by having a radius of less than ten times its wall thickness, capable of being inserted into a patient comprising a wire or a plurality of wires formed into a helix with closely connecting coils, having a distal and proximal end and an internal lumen, wherein said helix is shaped into a pigtail or a partial loop or a single loop or a plurality of loops at either the proximal end or the distal end or both the proximal and distal ends of said helix wherein said pigtail, partial loop, single loops or plurality of loops prevent the migration of the stent from the location said stent is implanted in a patient, and further wherein the proximal end of said stent is not longitudinally connected to the distal end of said stent.

The claimed stent may be modified with an atraumatic tip on either the proximal end or the distal end or both or wherein said wires are hollow or wherein the proximal ends or the distal ends or both the proximal and distal ends of the wires forming the helix ending in an anti-migration pigtail, partial loop, single loop or plurality of loops are braided or plated or welded together or attached to another anti-migration feature or wherein said helix is comprised of one coiled wire or a plurality of coiled wires consecutive coils used to form the helix touch each other or wherein said stent is coated or plated with silver or gold metal, a radiopaque material, an anti-bacterial coating, a drug, a polymer coating or is inserted in a tube or any combination thereof or wherein the plurality of coiled wires coiled together to form the helix of the stent transitions to an uncoiled section and that said uncoiled section further is covered with a tube or is uncovered and is reinforced with a mandrel or a cannula or is not reinforced or wherein said wires are solid wires or wherein said stent is covered by a polymer coating or is inserted in a tube or wherein said hollow wires, plastic coating or outer tube may further have a plurality of holes throughout the surface of said wires, coating or outer tube or wherein said stent is used to treat a urological condition or wherein said tube is manufactured from a tube or metallic tube or a hypotube used to treat bodily vessel obstructions, said stent being formed by a laser cutting said tube, metallic tube or hypotube into a helix.

The claimed stent may also further comprise a wire or rod or a plurality of wires or rods attached to the outer or inner surface of the helix of coiled wire or coiled wires or further comprising a spiral cup shape, circular disc shape or cup shape on top of an inverted cup shape formation at one or both ends.

In another embodiment, the claimed stent may be fitted with an outer sheath which comprises a tube having a proximal and distal end and an inner lumen extending throughout the entire length of said tube wherein when the stent is inserted into the proximal end of the lumen of said tube said stent is pushed along the length of the tube by the stent inserter and the guide wire of or wherein the outer sheath further comprises one or more radiopaque markers at the distal end of the sheath or wherein the outer sheath is coated with a hydrophilic coating or wherein the stent inserter is comprised of a single material or a composition of materials, or braided or coiled materials provided said stent inserter has a compressive strength greater than 50 kPa or wherein the stent inserter further comprises markings on indicating the length of the stent inserter from the distal end to the proximal end of the stent inserter, wherein when the stent is inserted into the patient, said markings indicate to the practitioner inserting the stent, the position of the stent in the patient's body as it is being inserted into the patient or wherein the distal end of the stent inserter is atraumatic or radiopaque or has radiopaque marker bands or wherein the stent inserter is surrounded by a tube that can be detachable or not detachable or where any surface of the inserter or sheath is coated with a lubricious coating or a hydrophilic coating or wherein the stent is inserted into the patient by pulling back the sheath with the stent inserter held in position until the sheath is pulled back and the proximal end of the stent is fully deployed, at which point the sheath is in line with the inserter and both sheath and inserter can be withdrawn from the body in conjunction with each other or separately or wherein the guide wire is an over-the-wire system, a long-wire system, a short-wire system or a wire system through a side port.

In another embodiment, the invention is directed to a method of placing a metallic stent in the ureter using a metallic hypotube as a delivery device, said hypotube comprising a metal tube with a lumen or a number of lumens and having a plurality of openings cut through or cut onto the tube surface to increase flexibility and said stent being pushed from the proximal end of the hypotube to the distal end of the hypotube for delivery within a body lumen of a patient, whereby the hypotube provides pushability and radial strength in narrow ureters.

The stent of the instant application possesses a suitable radial strength to enable bodily vessels to function regardless of any type of unwanted forces imposed on and in those vessels. It is manufactured from a material that allows for the stent to remain in place after insertion into the patient for an extended indwell duration of at least one year without complications when the stent is finally removed from the patient after such an extended period.

The stent of the instant application can also be produced in a range of sizes to fit all ureters, including dimensions that are significantly smaller than the prior art, thereby eliminating the possibility of damage and trauma to the patient. This is an obvious benefit to both physicians placing the stents and to patients receiving the stent.

There are structural differences between the stent design of the instant application and the stent designs found in the prior art, leading to functional differences that allow for the innovative stent of the instant application to function in very small and tortuous vessels.

For instance, the stent in this invention has no connection between the distal end and the proximal end other than by the coiled wire used to form the helix. The stent of the instant application exhibits a significantly lower compressive strength longitudinally than the stent described in the Lavelle patent. In addition, the claimed stent also exhibits significantly lower tensile strength than a hollow wire stent like the one described in the Lavelle patent or a plastic stent similar to the one claimed in U.S. Pat. No. 5,531,741 (“the Barbacci patent”). These features of the stent of this invention provide extreme flexibility, allowing for the use of the present stent in very tortuous vessels without causing trauma to the vessels.

The stent of the present application exerts force radially around the coil of the wire, keeping a vessel open against forces exerted by obstructions and strictures, thus allowing liquid to flow along it. Any longitudinal force exerted by having closely connecting coils lined up in a linear fashion would be dissipated by the fact that those coils are not connected longitudinally, causing the coils to separate and bend with the vessel, rather than causing trauma to the vessel or creating a perforation in the vessel. By closely connecting the coils, strictures or obstructions that block the inner lumen of the stent are prevented.

In addition, by not having any central rod or second inner lumen like the stents of the prior art, the stent of the instant application can be manufactured with much smaller dimensions than those stents with a central rod or second inner lumen. In a practical scenario, an adult domestic cat ureter is approximately twenty thousandths of an inch in diameter. Stent dimension is an issue that is central to the design of a stent for the cat ureter. Stents currently on the market and described in the prior art are incapable of being made to certain dimensions. The stent of the instant application can be designed to accommodate a wide range of sizes and dimensions thus addressing a long felt need in the art of stent design.

Another structural difference between the stent of the instant invention and stents described in the prior art is the use of solid wire. Prior art stents utilizing a hollow wire or plastic parts lack the strength that the instant stent exhibits. Solid wire is used in the claimed stent because it has much higher radial strength than hollow wire does and a stent can be manufactured in much smaller dimensions than can a stent using hollow wire. The stent of the instant application also has vastly greater radial strength than stents made of plastics.

The stent of the instant application can be used to assist urine flow in the ureter and the urethra, where issues have occurred to stem urine flow, but it can additionally be used in other parts of the body.

A preferred system of delivering the stent of the instant application to its intended location in the body of a patient is a minimally invasive delivery system. Without such a delivery system, the stent would have to be placed percutaneously, which should be avoided if possible. Percutaneous placement involves cutting open the body of the patient to place the stent which exposes the body to possible infection and which causes pain and slower recovery. While there are several delivery systems possible in larger versions of this invention, the need for compressive force to push the stent means that an over-the-wire system would not generally work in a 1.5 Fr stent kit, for example. This invention overcomes this area of difficulty. However, limitations in the physiology of some small animals will mean that percutaneous placement will be necessary in some cases.

One delivery system of the stent kit in this invention involves placing a sheath in the ureter by pushing it through the urethra and bladder and into the ureter with a stent inserter or guide wire inside it. The sheath placement allows for the stent to be pushed through the sheath by the inserter which allows for the stent inserter to be larger than the stent. Having as large a stent inserter as would fit the delivery system is essential given the dimensions of the component parts in the smaller versions of the stent and the fact that the inserter must be able to push past stones in the ureter, as part of the initial placement of the sheath. It is also essential to use materials of a high compressive strength for the same reason. The design of this invention allows for both necessities.

The stent itself would typically be made from coiled, solid wires for strength and would be coated or plated with a metallic substance, such as gold, for radiopacity and to prevent bacteria adhering to the surface of the stent and making its way from the bladder to the kidney, which could be thus infected. The coils of the stent are close together and would generally be touching adjacent coils, which provides further strength in the stent. This radial strength is needed to prevent deformation by stones or strictures in the ureter. The coils also provide a further essential function of allowing urine to squeeze into the coil from the ureter or renal pelvis and to drain through the lumen of the stent into the bladder. The coil continues into a pigtail or loop at the end of the stent, which gives the anchoring anti-migration pigtail or loop more strength. This strength is essential, given that some of the intended uses of this product are in stents of 1 Fr or less in outside diameter, which are made from wire of 0.003″, to facilitate placement in small animals and possible paediatric use.

The stent in this invention is designed for a tortuous ureter for use in veterinary and paediatric urology applications. Kinking forces present in the ureter after stent placement were taken into consideration when designing the stent in this invention, as kinking forces often cause stent failure. This is particularly apparent when the vessel is highly tortuous. The stent design, therefore, needs to be highly kink-resistant. A study conducted by Christman et al. (Christman M S et al., (ePub 2009), BJU Int, 105(6):866-9), on the kink resistance of ureteral stents showed that the larger the curvature of the stent, the greater the deformation possible before failure and the more resistant the stent is to kinking. From this study, it was demonstrated that larger diameter stents had less allowable curvature before failure than smaller diameter stents. Therefore, the stent with the smallest diameter had the greatest allowable curvature and was the most kink resistant. U.S. Pat. No. 6,395,021 references stent diameters of between ⅛″ (0.125″; 3.2 mm; 9 Fr) to ¼″ (0.250″; 6.3 mm; 19 Fr). The stent in this invention is designed with a diameter of between 0.013″-0.052″ (1 Fr-4 Fr). This design feature enables it to be highly kink-resistant in a tortuous anatomy and avoid stent failure. has been shown by Christman et al., 2009 that ureteral stent diameters of greater than 6 Fr are not kink-resistant.

Radial compression is often the cause of stent failure. The stent in this invention is designed to withstand high external forces from both intrinsic and extrinsic strictures. U.S. Pat. No. 6,395,021 (“the Hart patent”) describes a stent as a flexible duct having a very thin wall. The coiled stent design outlined in the Hart patent, See FIG. 8.0, is shown to be cylindrical in shape. The definition for a thin-walled cylindrical vessel is defined by the ratio of radius to wall thickness with a ratio greater than 10 required (r/t>10 or d/t>20; r=radius, t=wall thickness, d=diameter). The stent design proposed in this invention falls into the category of a thick-walled cylinder where the d/t ratio is <20, and is not considered to be a thin-walled vessel. The stent was designed as a thick-walled vessel to ensure it could withstand high radial forces, which are reported to be exerted on a stent by both intrinsic and extrinsic strictures, and therefore prevent stent failure.

It is reported that pressure in an obstructed ureter can reach 100 mm Hg (Kill F, ‘Urinary Flow and Ureteral Peristalsis,’ Urodynamics, (1^{ST} Ed, 1973), W. Lutzeyer and H Melchior (eds), Springer-Verlag Berlin Publishers, Heidelberg, DE (Pub), pp. 57-70). Based on this report, 100 mm Hg pressure was used as a possible pressure that ureteral stents can experience. These high pressures can cause a stent to buckle when in place, which can result in stent failure.

External stress exerted on a stent can result in stent failure. Hoop stress is a type of mechanical stress on a cylindrically-shaped part because of internal or external pressure. It can be defined as the average force exerted circumferentially (perpendicular both to the axis and to the radius) on every particle in the cylinder wall. Furthermore, hoop stress exerted over a walled vessel is also related to the D/t ratios. The formula for hoop stress exerted on a thin-walled, cylindrical vessel is:

Hoop Stress=P*D/2t

### P=Pressure

D=Mean diameter (outside diameter minus t)
t=wall thickness

The hoop stress formula for a thick-walled vessel falls under Lamé's equation:

$${σ}_{h} = \frac{{p}_{i} {r}_{i}^{2} - {p}_{o} {r}_{o}^{2}}{{r}_{o}^{2} - {r}_{i}^{2}} + \frac{( {p}_{i} - {p}_{o} ) {r}_{o}^{2} {r}_{i}^{2}}{( {r}_{o}^{2} - {r}_{i}^{2} ) {r}^{2}}$$

σₕ=Hoop stress

Pᵢ=internal pressure

Pₒ=external pressure

rᵢ=internal radius

rₒ=external radius

r=radius at point of interest (usually rᵢ or rₒ)

What is clear when applying the formula for hoop stress for both thin-walled and thick-walled cylinders, is that hoop stresses experienced on a thick-walled cylinder at the same pressure are significantly less than that observed for the thin-walled vessel. Therefore, at a given pressure, the thin-walled stent has a much greater chance of failure when exposed to external stresses.

Furthermore, the stent design in this invention may also have a wire or rod, or a plurality of same, attached to the outer surface or inner surface of the helix of coiled wire for given conditions such as angular blockages to further prevent stent failure. The wire or rod may be constructed of any material, which meets the design requirements, such as a metal or polymer or a combination of materials. The wire or rod could extend along the entire length of the helix or along a portion of the length of the helix. Attaching a wire to the inner or outer surface of the helix or a portion of the length of the helix allows for greater flexibility in the stent than attaching a wire through the centre of the lumen of the helix to end caps.

There would also be a reduction in tension on the wire used if it is attached to the inner or outer surface of the stent as opposed to being attached through the centre of the helix. A helix will naturally turn until it is in a state of lowest tension in accordance with the forces acting on it. That natural movement would be restricted by a wire passing through the centre of the helix and attached to end caps. Using a wire attached to the inner or outer surface of the helix reduces the possibility of said wire breaking due to excess tension and perforating the vessel.

Apart from the stent, the sheath used in the Applicant's invention is very different to that used in the references of the Lavelle patent and the Barbacci patent, which further emphasises the design and function differences needed in the stent. The sheath would typically be a metal hypotube, laser-cut with a spiral cut or interrupted cut design.

Metal would generally be used in the hypotube for its radial strength and for the fact that it can provide substantial radial strength from a very thin wall thickness. Metal also has high compressive strength, which increases pushability. Use of a sheath gives a clear path for deployment of the stent of this invention whose pushability is dependent on its closely connecting multifilar coil, rather than a more pushable rigid body connecting the proximal end to the distal end, as taught in the Lavelle stent.

### BRIEF DESCRIPTION OF THE DRAWINGS

FIG. 1a is an illustration of prior art and FIG. 1b is an illustration of prior art;

FIG. 2 is an illustration of the inserter;

FIG. 3a is a cross-sectional view of the stent inserter with the mandrel placed inside it;

FIG. 3b is a cross-sectional view of the stent inserter without any mandrel inside it;

FIG. 4 is an illustration of the outer sheath;

FIG. 5 is an illustration of the radiopaque tip of the sheath and the stent inserter;

FIG. 6 is an illustration of the sheath with the stent inserter inside it;

FIG. 7 is an illustration of the cross-sectional view of the sheath;

FIG. 8 is an illustration of the markings on the proximal end of the inserter;

FIG. 9 is an illustration of a coiled pig-tailed stent;

FIG. 10 is an illustration of a longitudinal section of the coiled stent;

FIG. 11 is an illustration of a coiled stent with a pig-tail on one end only;

FIG. 12 is an illustration of a cross-sectional view of the stent;

### DETAILED DESCRIPTION OF THE INVENTION

One embodiment of the present invention is a kit for placing a stent. This kit will typically include an outer sheath, stent inserter, stent and a stent holder. The sheath and inserter are designed to be used in combination according to user requirements and are used solely for placement of the indwelling stent and removed after this has been achieved.

The stent in this embodiment is typically made from metal wires which have been coiled over a mandrel to create a cylindrical shaped part possessing an internal lumen, which makes up the body of the stent. The ends of the stent would typically be formed into a loop or a pigtail at the distal and proximal ends. The wire of the stent will typically be terminated by laser welding the wire into the end or by welding the wire beneath a ball or similar object to create an atraumatic surface.

There are numerous possible embodiments associated with the stent kit and stent design for the invention described herein and these are detailed in the claims below. All drawings, summaries, descriptions, embodiments and objects are intended to be illustrative rather than limiting.

### Embodiments

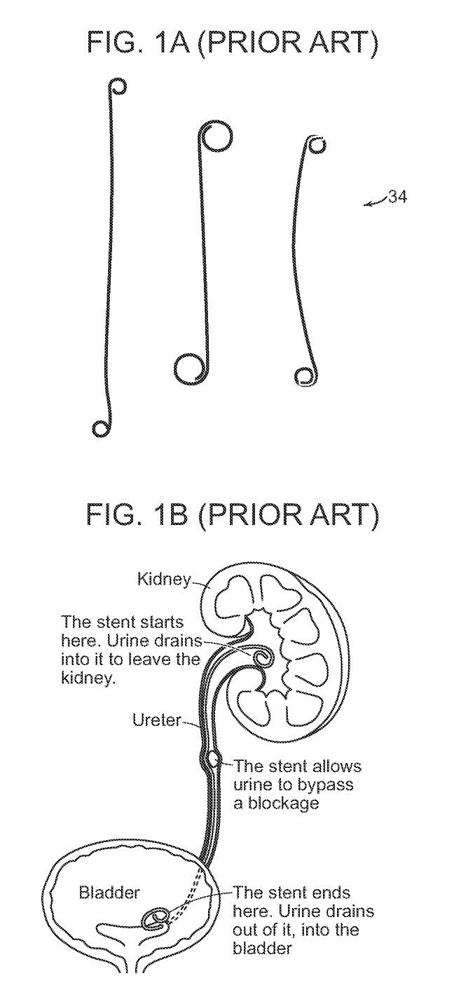

FIG. 1A shows prior art of ureteral stents 34. FIG. 1B shows the function of ureteral stents. Typically, these stents are placed in a minimally invasive manner by passing the stent over a guide wire that has been positioned in the renal pelvis of the kidney. A pusher is used to advance the stent along the wire from the urethra to the bladder and subsequently into the ureter. Other methods for stent placement are percutaneously where the physician accesses the ureter through the skin of patient using nephrostomy methods.

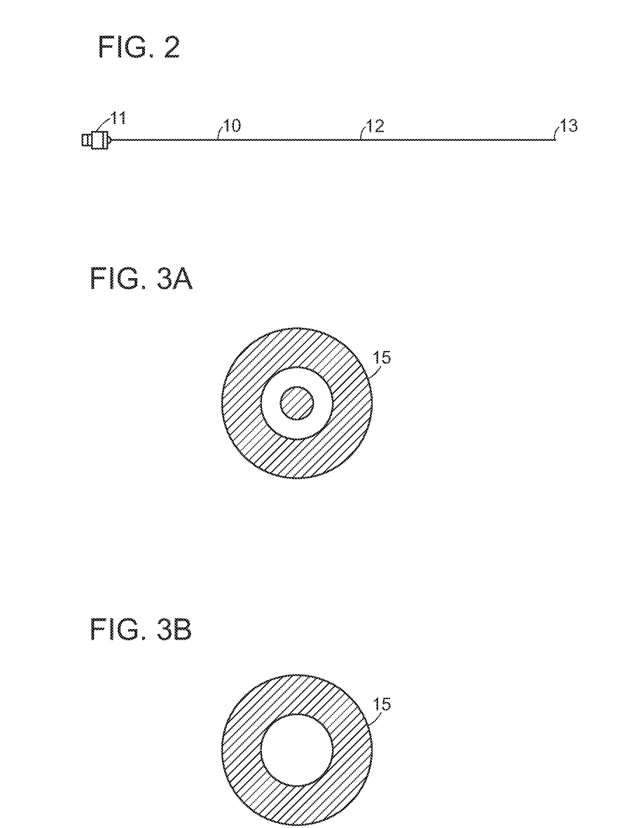

FIG. 2 displays the stent inserter and outer sheath. An embodiment of the design detailed in FIG. 2 shows the small diameter of the outer sheath. The stent inserter reference number in FIG. 2 is 10. This significantly reduces trauma to the patient during the procedure and is specifically suitable for patients with ureters that are sized in the range from 0.3 mm to 0.7 mm (0.011″-0.027″). Other physician benefits are the highly radiopaque properties of the stent and the inserter tip allowing the physician to place the device without the need for a guide wire. This would be a benefit during paediatric or small animal veterinary procedures where it is important to keep fluoroscopic exposure to a minimum.

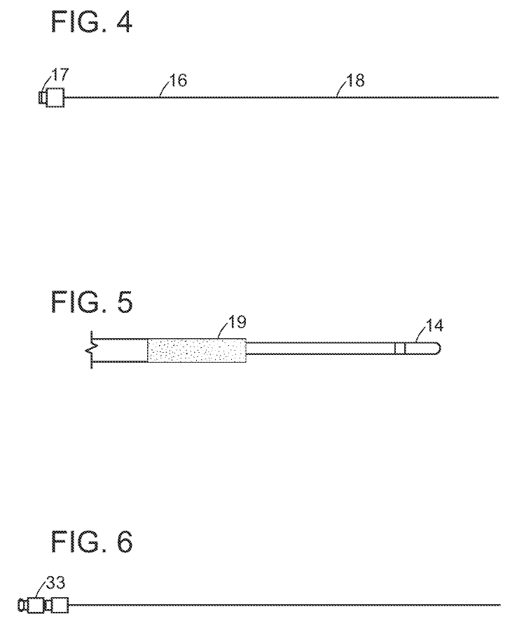

A sheath incorporating one embodiment of the invention is generally indicated by the reference number 16 in FIG. 4.

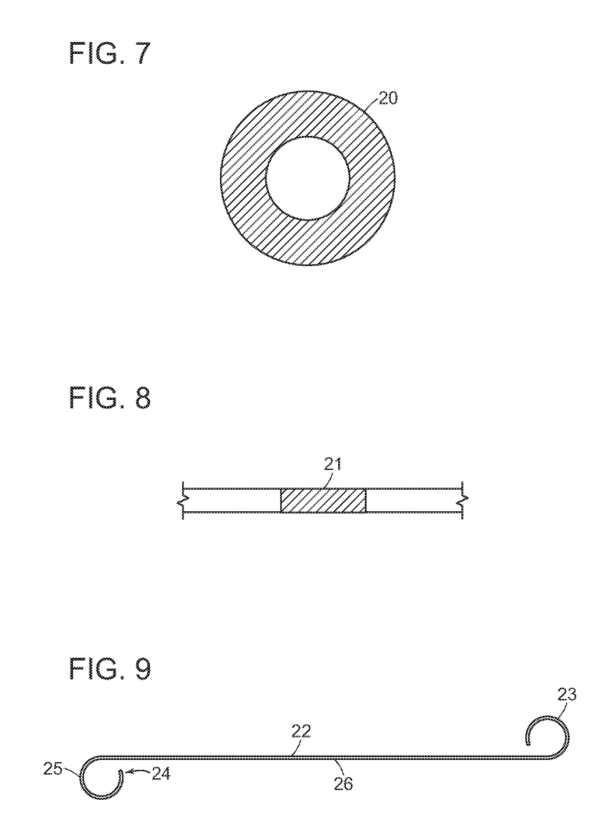

Materials most suitable for the outer sheath might be stainless steel, PTFE, FEP, fluoropolymer, silicone, polyurethane, polyethylene, PEBAX and nylon, but any material approved for use in medical devices can be used. The sheath would typically be flared at the proximal end. The flared end can be attached to the connector cap or handle by gluing or by over-moulding or by welding the proximal end on to a connector cap or handle 17 (FIG. 4). The connector cap could have a male ending to enable it to attach to or detach from the female luer 33 (FIG. 6) of the inserter. The distal end has a radiopaque marker as seen in FIG. 5, reference number 19. The radiopaque marker would typically be a marker band, a radiopaque filler encompassed in the plastic of the sheath or radiopaque ink. The dimensions of the sheath would typically be 15 cm to 60 cm in length 18 (FIG. 4) and typically 0.011″ to 0.090″ in diameter 20 (FIG. 7).

The inserter 10 (FIG. 2) can consist of a metal or a plastic polymer material such as, but not restricted to PVC, polyurethane, polyethylene, silicone, FEP, PEBAX, polyamide, polyimide and PEEK. Alternatively, the stent inserter can be a combination of a polymer tube surrounding a metal cannula or mandrel (FIG. 3a). The dimensions of the inserter 10 would typically be 15 cm to 60 cm in length and typically 0.007″-0.090″ in diameter 15 (FIG. 3a & FIG. 3b). The stent inserter could have markings 21 (FIG. 8) on its length 12 (FIG. 2) to denote distance of the distal end of the inserter within the outer sheath and/or to give indication of the stent and/or inserter position during the procedure.

If the inserter was to be made of a cannula or mandrel surrounded by a polymer tube, the metal part may be the same length as the polymer tube 13 or it could be shorter than the tube to allow for a polymer atraumatic tip. Alternatively, the metal inserter could be shaped to form an atraumatic tip. The metal inner section may also be ground or electro-polished to taper at the distal end to create an atraumatic tip 14 (FIG. 5).

For successful placement of the inserter tip in the renal pelvis of the kidney, it is essential that the distal tip of the inserter is highly radiopaque 14. This can be achieved by: incorporating a radiopaque filler in the distal section of the polymer inserter; a radiopaque metal maker band; radiopaque ink; metal plating with a radiopaque metal; or a radiopaque polymer strip embedded in the distal tip during processing. The metal part of the inserter can be attached to the outer inserter tube via a luer and a connector cap 11. The polymer end of the inserter would typically be flared with a female luer end to allow the physician to attach or detach it from the outer sheath.

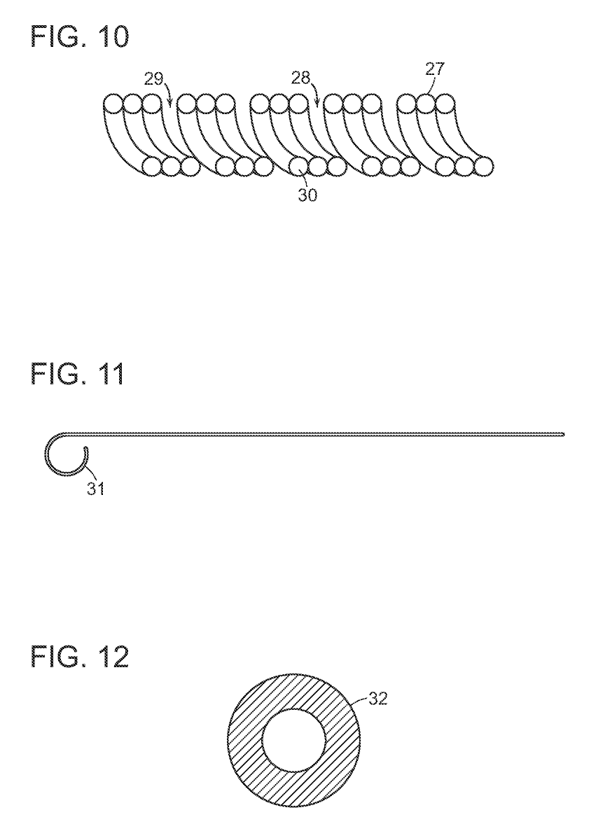

An embodiment of the design detailed in FIG. 9 is a coiled stent 22. The stent can be produced by coiling a metal wire such as stainless steel, nitinol, MP35N or MP159 or by using a mix of metal and a polymer. The coil pattern can be any hilar pattern or a variety of hilar patterns 28 (FIG. 10).

Pressure generated in the kidney and ureter would force urine in through the coils 29 (FIG. 10) of the stent and it could drain into the bladder through the coils in the proximal end of the stent.

The stent would typically have a looped pigtail 23 on both the distal and proximal ends to prevent migration of the stent from its position in the ureter. The loops would be created by heat-forming the metal used in the stent. The diameter 25 of the loop/pigtail can be in the range of 3-16 mm.

It is also possible for the stent to have a straight body and only transition into a loop or pigtail on one end 31 (FIG. 11). The stent can also be produced by a braided mesh and with polymer covering/membrane over the mesh.

Other additions to the stent design could consist of a polymer coating or membrane over the stent. Holes could be created in the membrane of the stent to allow urine to flow into or out of the stent, according to the pressure exerted. The holes could be created by perforating the membrane with a sharp tool or by laser.

The wire used to create the stent would typically range in size from 0.0005″ to 0.040″ 30 (FIG. 10).

The wire can be any produced in various shapes. The diameter of the stent would typically be from 0.3 Fr to 5.0 Fr, as seen at reference 32 (FIG. 12). The length of the stent would typically range from 3 cm to 20 cm, as seen at reference 26 (FIG. 9).

Alternatively, the stent could be created by laser cutting a hollow tube and forming it into the required shape. The laser cutting could begin with a hypotube, HHS, mandrel or cannula to form the required shape. The thickness of the metal tube used to create the stent could range from 0.0005″ to 0.040″. The laser could cut out a single design iteration or a variety of designs.

The tip of the stent 24 (FIG. 9) could be created by soldering or laser welding the end of the wire into the end of stent or by welding a radiopaque dome-shaped cap or ball on the ends of the stent or by welding on a plastic or polymer tip. The intention regarding the tip 24 is that it would be atraumatic to the body of the patient and biocompatible. The stent can be plated with a metallic material, such as gold, to enhance its radiopaque properties, prevent encrustation and to provide a smooth surface, onto which bacteria cannot easily adhere. Other forms of finishing would include passivation, mechanical polishing, etching, slurry cleaning the internal diameter and electro-polishing. Anti-bacterial or anti-encrustation coating may also be applied to the stent's surface 27 (FIG. 10) to prevent or reduce encrustation of the stent for its indwell duration.

What is claimed is:

1. A thick-walled stent, as defined by having a radius of less than ten times its wall thickness, capable of being inserted into a patient comprising a wire or a plurality of wires formed into a helix with closely connecting coils, having a distal and proximal end and an internal lumen, wherein said helix is shaped into a pigtail or a partial loop or a single loop or a plurality of loops at either the proximal end or the distal end or both the proximal and distal ends of said helix wherein said pigtail, partial loop, a single loop or plurality of loops prevent the migration of the stent from the location said stent is implanted in a patient, and further wherein the proximal end of said stent is not longitudinally connected to the distal end of said stent.

2. The stent of claim 1, with an atraumatic tip on either the proximal end or the distal end or both or wherein said wires are hollow or wherein the proximal ends or the distal ends or both the proximal and distal ends of the wires forming the helix ending in an anti-migration pigtail, partial loop, a single loop or a plurality of loops are braided or plated or welded together or attached to another anti-migration feature or wherein said helix is comprised of one or more plurality of coiled wires consecutive coils used to form the helix touch each other or wherein said stent is coated or plated with silver or gold metal, a radiopaque material, an anti-bacterial coating, a drug, a polymer coating or is inserted into a tube or any combination thereof or wherein the plurality of wires coiled together to form the helix of the stent transitions to an uncoiled section and that said uncoiled section further is covered with a tube or is uncovered and is reinforced with a mandrel or a cannula or is not reinforced or wherein said wires are solid wires or wherein said stent is covered by a polymer coating or is inserted in a tube or wherein said hollow wires, plastic coating or outer tube may further have a plurality of holes throughout the surface of said wires, coating or outer tube or wherein said stent is used to treat a urological condition or wherein said tube is manufactured from a tube or metallic tube or a hypotube used to treat bodily vessel obstructions, said stent being formed by a laser cutting said tube, metallic tube or hypotube into a helix.

3. The stent of claim 1, further comprising a wire or rod or a plurality of wires or rods attached to the outer or inner surface of the helix of coiled wire or coiled wires or further comprising a spiral cup shape, circular disc shape or cup shape on top of an inverted cup shape formation at one or both ends.

4. The stent of claim 1 incorporated in a stent kit composed of an outer sheath, a stent inserter or a guide wire, a stent loading tube, ancillary parts and the stent according to claim 1.

5. The stent kit according to claim 4, wherein the outer sheath comprises a tube having a proximal and distal end and an inner lumen extending throughout the entire length of said tube wherein when the stent is inserted into the proximal end of the lumen of said tube said stent is pushed along the length of the tube by the stent inserter and the guide wire of claim 4 or wherein the outer sheath further comprises one or more radiopaque markers at the distal end of the sheath or wherein the outer sheath is coated with a hydrophilic coating or wherein the stent inserter is comprised of a single material or a composition of materials, or braided or coiled materials provided said stent inserter has a compressive strength greater than 50 kPa or wherein the stent inserter further comprises markings on indicating the length of the stent inserter from the distal end to the proximal end of the stent inserter, wherein when the stent is inserted into the patient, said markings indicate to the practitioner inserting the stent, the position of the stent in the patient's body as it is being inserted into the patient or wherein the distal end of the stent inserter is atraumatic or radiopaque or has radiopaque marker bands or wherein the stent inserter is surrounded by a tube that can be detachable or not detachable or where any surface of the inserter or sheath is coated with a lubricious coating or a hydrophilic coating or wherein the stent is inserted into the patient by pulling back the sheath with the stent inserter held in position until the sheath is pulled back and the proximal end of the stent is fully deployed, at which point the sheath is in line with the inserter and both sheath and inserter can be withdrawn from the body in conjunction with each other or separately or wherein the guide wire is an over-the-wire system, a long-wire system, a short-wire system or a wire system through a side port.

6. A method of placing a metallic stent in the ureter using a metallic hypotube as a delivery device, said hypotube comprising a metal tube with a lumen or a number of lumens and having a plurality of openings cut through or cut onto the tube surface to increase flexibility and said stent being pushed from the proximal end of the hypotube to the distal end of the hypotube for delivery within a body lumen of a patient, whereby the hypotube provides pushability and radial strength in narrow ureters.
